(19) United States
(12) Patent Application Publication
(10) Pub. No.: US 2024/0090834 A1
(43) Pub. Date: Mar. 21, 2024

(54) Wearable Computing Device Having One or More Biometric Sensor Electrodes on a Cover for a Display Screen

(71) Applicant: Fitbit LLC, San Francisco (CA)

(72) Inventors: Kevin Li, San Diego, CA (US); Ritu Verma, San Diego, CA (US)

(21) Appl. No.: 18/013,711

(22) PCT Filed: Oct. 7, 2021
(86) PCT No.: PCT/US2021/053927

§ 371 Date: Dec. 29, 2022

(51) Int. Cl. A61B 5/00; G04G 21/02; G04G 21/04; G06F 1/16; G16H 40/67; H01Q 1/27

(52) U.S. Cl. CPC A61B 5/681; A61B 5/0022; A61B 5/742; G04G 21/025; G04G 21/04; G06F 1/163; G16H 40/67; H01Q 1/273

(57) ABSTRACT

A wearable computing device is provided. The wearable computing device includes a, conductive housing and a printed circuit board at least partially disposed within the conductive housing. The wearable computing device further includes a slot antenna defined by a gap between the conductive housing and the printed circuit board. The wearable computing device includes a display screen electrically coupled to the printed circuit board. The wearable computing device includes a cover positioned on the display screen. The cover includes a top surface and a bottom surface. The wearable computing device includes a biometric sensor electrode positioned partially on the top surface of the cover. The biometric sensor at least partially wraps around a periphery of the cover.

### FIELD

The present disclosure relates generally to wearable computing devices. More particularly, the present disclosure relates to a wearable computing device having a biometric sensor electrode positioned partially on a top surface of a cover for a display screen of a wearable computing device.

### BACKGROUND

Wearable computing devices (e.g., wrist watches) can include a display screen to display content (e.g., time, date, etc.) to a user. Wearable computing devices can include one or more antennas and transceivers. In this manner, wearable computing devices can communicate with other devices (e.g., smartphones). Wearable computing devices can gather data regarding activities performed by the user, or regarding the user's physiological state. Such data may include data representative of the ambient environment around the user or the user's interaction with the environment. For example, the data can include motion data regarding the user's movements and/or physiological data obtained by measuring various physiological characteristics of the user, such as heart rate, perspiration levels, and the like.

### SUMMARY

Aspects and advantages of embodiments of the present disclosure will be set forth in part in the following description, or can be learned from the description, or can be learned through practice of the embodiments.

In one aspect, a wearable computing device is provided. The wearable computing device includes a conductive housing and a printed circuit board at least partially disposed within the conductive housing. The wearable computing device further includes a slot antenna defined by a gap between the conductive housing and the printed circuit board. The wearable computing device includes a display screen electrically coupled to the printed circuit board. The wearable computing device includes a cover positioned on the display screen. The cover includes a top surface and a bottom surface. The wearable computing device includes a biometric sensor electrode positioned partially on the top surface of the cover. The biometric sensor at least partially wraps around a periphery of the cover.

In some implementations, the biometric sensor electrode a sheet resistance such that the biometric sensor electrode is at least partially radio frequency transparent at a frequency at which the slot antenna is operable. In some implementations, the sheet resistance is about 3000 ohms per square for the frequency at which the slot antenna is operable.

In some implementations, the biometric sensor electrode has a sheet resistance such that the biometric sensor electrode supports radio frequency currents for loading the slot antenna at a frequency at which the slot antenna is operable. In some implementations, the sheet resistance is less than about 200 ohms pers square for the frequency at which the slot antenna is operable.

In some implementations, a gap is defined between an active display area of the display screen and the biometric sensor electrode. For instance, in some implementations, a width of the gap ranges from about 0.5 millimeters to about 3 millimeters.

In some implementations, the cover includes an optically transparent material. For instance, in some implementations, the optically transparent material includes a glass material.

In some implementations, the wearable computing device includes an electrical contact disposed on the bottom surface of the cover. Furthermore, the biometric sensor electrode contacts the electrical contact on the bottom surface of the cover.

In some implementations, at least a portion of the biometric sensor electrode includes a physical vapor deposition (MID) defined coating. For instance, in some implementations, the portion of the biometric sensor electrode includes at least a first portion positioned on the top surface of the cover or a second portion positioned on the bottom surface of the cover.

In some implementations, the top surface of the cover and the bottom surface of the cover are flat. Furthermore, in some implementations, the periphery of the cover includes a curved surface.

In another aspect, a wearable computing device is provided. The wearable computing device includes a conductive housing and a printed circuit board at least partially disposed within the conductive housing. The wearable computing device further includes a slot antenna defined by a gap between the conductive housing and the printed circuit board. The wearable computing device includes a display screen electrically coupled to the printed circuit board. The wearable computing device includes a cover positioned on the display screen. The cover includes a top surface and a bottom surface. The wearable computing device includes a first biometric sensor electrode and a second biometric sensor electrode. The first biometric sensor electrode and the second biometric sensor electrode are each positioned partially on the top surface of the cover such that the first biometric sensor electrode and the second biometric sensor electrode are spaced apart from one another. The first biometric sensor electrode wraps around a first portion of a periphery of the cover. The second biometric sensor electrode wraps around a second portion of the periphery of the cover. Furthermore, the first biometric sensor electrode has a sheet resistance such that the first biometric sensor electrode is at least partially radio frequency transparent at a frequency at which the slat antenna is operable.

In yet another aspect, a wearable computing device is provided. The wearable computing device includes a conductive housing and a printed circuit board at least partially, disposed within the conductive housing. The wearable computing device further includes a slot antenna defined by a gap between the conductive housing and the printed circuit board. The wearable computing device includes a display screen electrically coupled to the printed circuit board. The wearable computing device includes a cover positioned on the display screen. The cover includes a top surface and a bottom surface. The wearable computing device includes a first biometric sensor electrode and a second biometric sensor electrode. The first biometric sensor electrode and the second biometric sensor electrode are each positioned partially on the top surface of the cover such that the first biometric sensor electrode and the second biometric sensor electrode are spaced apart from one another. The first biometric sensor electrode wraps around a first portion of a periphery of the cover. The second biometric sensor electrode wraps around a second portion of the periphery of the cover. Furthermore, the first biometric sensor electrode has a sheet resistance such that the first biometric sensor electrode supports radio frequency currents for loading the slot antenna at a frequency at which the slot antenna is operable.

These and other features, aspects, and advantages of various embodiments of the present disclosure will become better understood with reference to the following description and appended claims. The accompanying drawing, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 depicts a wearable computing device according to some implementations of the present disclosure.

FIG. 2 depicts an exploded view of a wearable computing device according to some implementations of the present disclosure.

FIG. 3 depicts a cross-sectional view of a wearable computing device according to some implementations of the present disclosure,

FIG. 4 depicts a slot antenna defined by a gap between a conductive housing of a wearable computing device and a printed circuit board of the wearable computing device according to some implementations of the present disclosure.

FIG. 5 depicts an exploded view of a portion of a wearable computing device according to some implementations of the present disclosure.

FIG. 6A depicts a side view of a cover for a display screen with a first biometric sensor electrode positioned partially on a top surface of the cover and wrapping at least partially around a periphery of the cover according to some implementations of the present disclosure.

FIG. 6B depicts a side view of a cover for a display screen with a second biometric sensor electrode positioned partially on a top surface of the cover and wrapping at least partially around a periphery of the cover according to some implementations of the present disclosure.

FIG. 7 depicts a top view of a wearable computing device according to some implementations of the present disclosure.

FIG. 8 depicts the top view of FIG. 7 with a cover, bands and housing of the wearable computing device removed according to some implementations of the present disclosure.

FIG. 9 depicts a bottom view of a cover of a wearable computing device according to some implementations of the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Example aspects of the present disclosure are directed to a wearable computing device that can be worn on a user's wrist. A wearable computing device can include a slot antenna defined by a gap (e.g., about 0.5 mm to about 10 mm) between a conductive housing (e.g., metal housing) of the wearable computing device and a printed circuit board of the wearable computing device. The wearable computing device can further include a display screen. In this manner, the display screen can display content (e.g., time, date, etc.) for viewing by the user. The wearable computing device can include a cover positioned on a top surface of the display screen to protect the display screen from being scratched. The cover can be optically transparent so that the user can view information being displayed on the display screen. As will be discussed below, the wearable computing device can include one or more biometric sensor electrodes positioned on the cover.

The one or more biometric sensor electrodes can be positioned partially on a portion of a top surface of the cover. Furthermore, the one or more biometric sensor electrodes can at least partially wrap around a periphery (e.g., edge) of the cover to contact one or more electrical contacts on a bottom surface of the cover. In this manner, the one or more biometric sensor electrodes can hide from view a portion (e.g., dead band) of the area surrounding an active area of the display screen. It should be understood that the area surrounding the active area of the display screen is referred to as a “dead band” of the display screen. Furthermore, since the one or more biometric sensor electrodes can be used to hide from view at least a portion of the dead band of the display screen, a separate decorative element (e.g., bezel) is not needed to cover the portion of the display screen to provide visual enhancement.

However, placement of the one or more biometric sensor electrodes on the cover can degrade performance of the slot antenna. For instance, the one or more biometric sensor electrodes can interfere with or degrade a radiation pattern associated with the slot antenna and/or a radiation efficiency of the slot antenna. As will be discussed below, the one or more biometric sensor electrodes can be configured so as to reduce or eliminate a degradation in performance of the slot antenna due to placement of the one or more biometric sensor electrodes on the cover.

In some implementations, at least a portion of the one or biometric sensor electrodes can have a first sheet resistance value such that the one or more biometric sensor electrodes are radio frequency transparent for a range of frequencies at which the slot antenna is operable. In some implementations, a radio frequency transparency of the at least a portion of the one or more biometric sensor electrodes can be at least 80% or at least 90% (i.e. a transmission coefficient of at least 0.8 or at least 0.9) for the range of frequencies at which the slot antenna is operable. For example, the first sheet resistance value can include any sheet resistance value that is greater than 2000 ohms per square for the range of frequencies (e.g., about 0.6 Gigahertz to about 10 Gigahertz) at which the slot antenna is operable. In this manner, degradation in the performance of the slot antenna over the range of frequencies due to placement of the one or more biometric sensor electrodes on the cover can be reduced or eliminated, because the one or more biometric sensor electrodes can be at least partially radio frequency transparent for the range of frequencies at which the slot antenna is operable.

In some implementations, the one or more biometric sensor electrodes can have a second sheet resistance value that is less than the first sheet resistance value so that the one or more biometric sensor electrodes can support radio frequency currents for loading the slot antenna over the range of frequencies at which the slot antenna is operable. For example, the second sheet resistance value can be less than about 200 ohms per square for the range of frequencies. In this manner, degradation in the performance of the slot antenna over the range of frequencies due to placement of the one or more biometric sensor electrodes on the cover can be reduced or eliminated, because the one or more biometric sensor electrodes support radio frequency currents for loading the slot antenna over range of frequencies at which the slot antenna is operable. In some implementations, the second sheet resistance value can be less than about 100 ohms per square. For instance, the second sheet resistance value can range from about 20 ohms per square to about 40 ohms per square.

In some implementations, at least a portion of the one or more biometric sensors can include a physical vapor deposition (PVD) coating. In such implementations, a thickness of a conductive material of the one or more biometric sensor electrodes can be limited due, at least in part, to the PVD coating. Furthermore, in such implementations, the sheet resistance of the one or more biometric sensor electrodes can be limited due, at least in part, to the thickness of the conductive material of the one or more biometric sensor electrodes being limited by the PVD coating process. More particularly, how low the sheet resistance of the one or more biometric sensor electrodes can be is limited.

A wearable computing device according to example aspects of the present disclosure can provide numerous technical effects and benefits. For instance, placing the one or more biometric sensor electrodes on the top surface of the cover (e.g., glass) for the display screen eliminates the need for a separate element (e.g., bezel) to cover the dead band of the display screen. Additionally, having the one or more biometric sensor electrodes on the top surface of the cover can allow for on-demand measurement of biometrics (e.g. electrocardiogram, electrodermal activity, etc.) of the user wearing the wearable computing device. Furthermore, the one or more biometric sensor electrodes can be configured to reduce or eliminate interference with a radiation pattern associated with the slot antenna of the wearable computing device at relevant frequency bands for communication and functionality of the wearable computing device. For instance, in some implementations, the sheet resistance of the one or more biometric sensor electrodes can be configured so that the one or more biometric sensor electrodes are radio frequency transparent for the relevant frequency bands. In alternative implementations, the sheet resistance of the one or more biometric sensor electrodes can be configured so that the one or more biometric sensor electrodes support radio frequency currents for the relevant frequency bands.

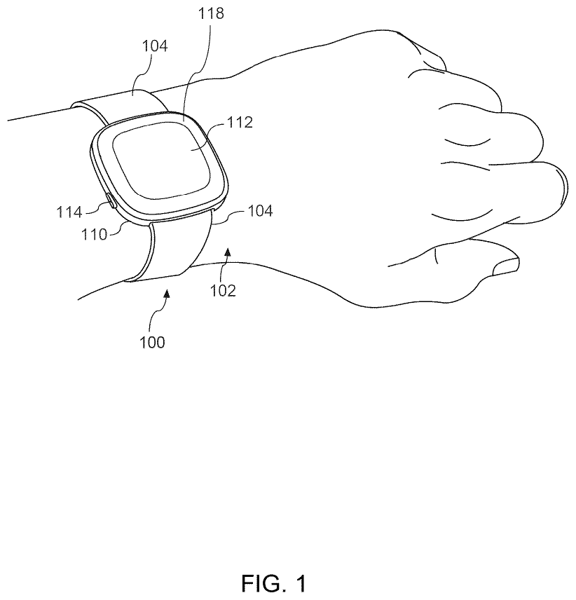

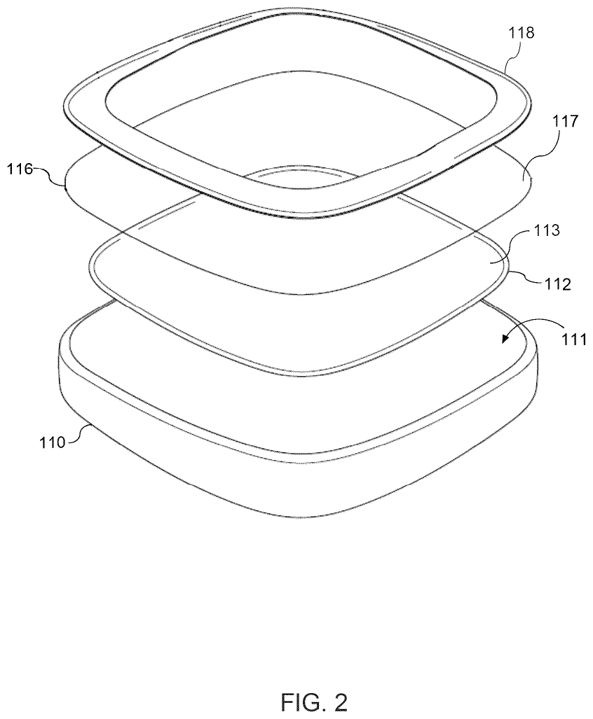

Referring now to the FIGS., FIGS. 1 and 2 depict a wearable computing device 100 according to some implementations of the present disclosure. As shown, the wearable computing device 100 can be worn, for instance, on an arm 102 (e.g., wrist) of a user. For instance, the wearable computing device 100 can include a band 104 and a conductive housing 110. It should be understood that the conductive housing 110 can include any suitable conductive material (e.g., metal).

The conductive housing 110 can be coupled to the band 104. In this manner, the band 104 can be fastened to the arm 102 of the user to secure the conductive housing 110 to the arm 102 of the user. Furthermore, the conductive housing 110 can define a cavity 111 for one or more electronic components (e.g., disposed on printed circuit boards) of the wearable computing device 100.

The wearable computing device 100 can include a display screen 112. The display screen 112 can display content (e.g., time, date, biometrics, etc.) for viewing by the user. In some implementations, the display screen 112 can include an interactive display screen (e.g., touchscreen or touch-free screen). In such implementations, the user can interact with the wearable computing device 100 via the display screen 112 to control operation of the wearable computing device 100.

In some implementations, the wearable computing device 100 can include one or more input devices 114 that can be manipulated (e.g., pressed) by the user to interact with the wearable computing device 100. For instance, the one or more input devices 114 can include a mechanical button that can be manipulated (e.g., pressed) to interact with the wearable computing device 100. In some implementations, the one or more input devices 114 can be manipulated to control operation of a backlight (not shown) associated with the display screen 112. It should be understood that the one or more input device 114 can be configured to allow the user to interact with the wearable computing device 100 in any suitable manner. For instance, in some implementations, the one or more input devices 114 can be manipulated by the user to navigate through content (e.g., one or more menu screens) displayed on the display screen 112.

The wearable computing device 100 can include a cover 116 positioned on the conductive housing 110 so that the cover 116 is positioned on top of the display screen 112. In this manner, the cover 116 can protect the display screen 112 from being damaged (e.g., scratched). In some implementations, the wearable computing device 100 can include a seal (not shown) positioned between the cover 116 and the conductive housing 110. For instance, a first surface of the seal can contact the cover 116 and a second surface of the seal can contact the conductive housing 110. In this manner, the seal between the conductive housing 110 and the cover 116 can prevent a liquid (e.g., water) from entering the cavity 111 of the conductive housing 110.

The cover 116 can be optically transparent so that the user can view information being displayed on the display screen 112. For instance, in some implementations, the cover 116 can include a glass material. It should be understood, however, that the cover 116 can include any suitable optically transparent material.

The cover 116 can be sized to cover a top surface 113 of the display screen 112. Furthermore, the wearable computing device 100 can include one or more biometric sensor electrodes 118 (only one shown) partially positioned on a portion of a top surface 117 of the cover 116. More particularly, the portion of the top surface 117 can include an outermost portion (e.g., periphery) of the top surface 117. In this manner, the one or more biometric sensor electrodes 118 can eliminate the need for having a separate element, such as a bezel, to frame the display screen 112. Furthermore, since the one or more biometric sensor electrodes 118 are disposed on the top surface 117 of the cover 116, the user can contact (e.g., touch) the one or more biometric sensor electrodes 118 to perform on-demand biometric measurements (e.g., electrocardiogram, electrodermal activity, etc.).

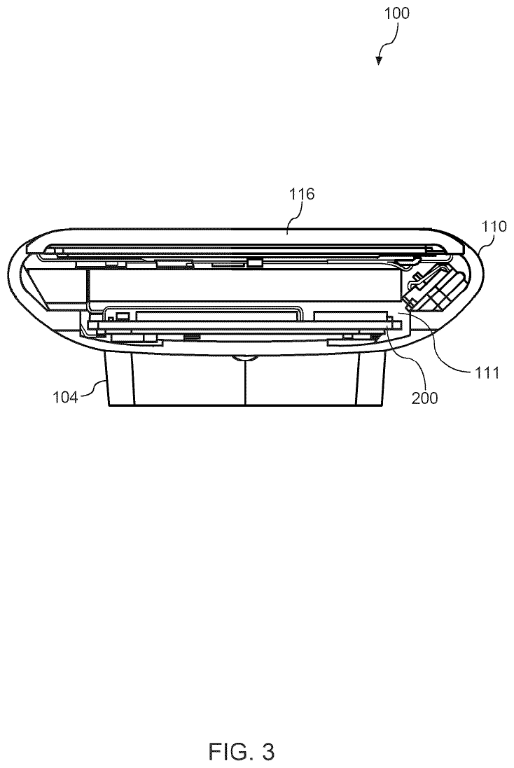

Referring now to FIG. 3, a cross-sectional view of the wearable computing device 100 is provided according to some implementations of the present disclosure. As shown, the wearable computing device 100 can include a printed circuit board 200 at least partially positioned within the cavity 111 defined by the conductive housing 110. It should be understood that the display screen 112 (FIG. 2) can, in some implementations, be electrically coupled to the printed circuit board 200 via a flexible printed circuit. Details of the printed circuit board 200 will now be discussed.

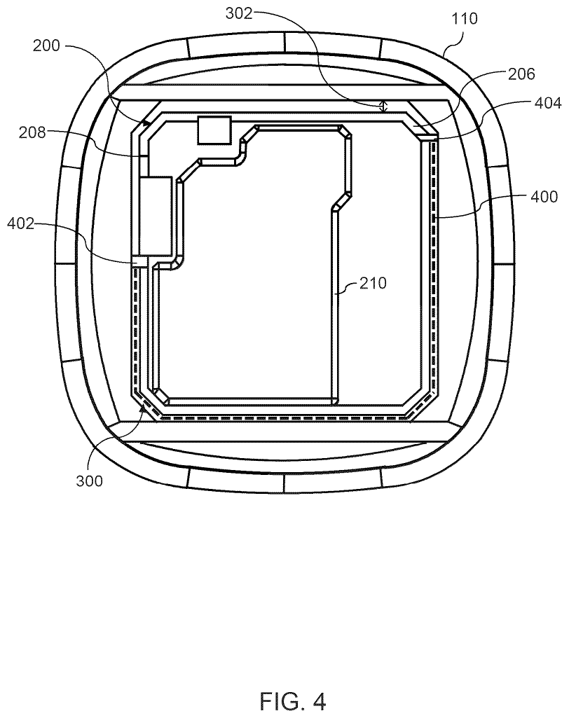

Referring now to FIG. 4, the printed circuit board 200 can be positioned relative to the conductive housing 110 such that a gap 300 is defined between the conductive housing 110 and the printed circuit board 200. More particularly, the gap 300 can extend from an interior surface of the conductive housing 110 to a periphery (e.g., edge) of the printed circuit board 200. Furthermore, in some implementations, the gap 300 can extend around the entire perimeter of the printed circuit board 200. Stated another way, in some examples, no edge of the printed circuit board 200 contacts (e.g., touch) the conductive housing 110.

In some implementations, a width 302 of the gap 300 defined between the conductive housing 110 and the printed circuit board 200 can range from about 0.5 millimeters to about 10 millimeters, Alternatively, or additionally, the width 302 of the gap 300 can, in some implementations, vary around the perimeter of the printed circuit board 200. For instance, the width 302 of the gap 300 between the conductive housing 110 and the printed circuit board 200 at a first portion of the perimeter of the printed circuit board 200 can be different (e.g., wider, narrower) than the width 302 of the gap 300 between the conductive housing 110 and the printed circuit board 200 at a second portion of the perimeter of the printed circuit board 200.

In some implementations, the perimeter of the printed circuit board 200 can include a copper free or around keep-out region 206. It should be understood that the ground keep-out region 206 can include a region of the printed circuit board 200 where electronic components (e.g., resistors, capacitors, etc.) cannot be placed. In some implementations, a width 208 of the ground keep-out region 206 of the printed circuit board 200 can range from 0.1 millimeters to about 2 millimeters. In alternative implementations, the printed circuit hoard 200 may not include the ground keep-out region 206. As will be discussed below, the ground keep-out region 206 can act as an electrical gap.

In some implementations, a slot antenna 400 (denoted by dashed line) can be defined by the gap 300 between the conductive housing 110 and the printed circuit board 200. Furthermore, in some implementations, the slot antenna 400 can be further defined by an electrical gap that spans the width 208 of the ground keep-out region 206 of the printed circuit board 200. In such implementations, the width of the slot antenna 400 can span the width 302 of the gap 300 and the width 208 of the ground keep-out region 206 of the printed circuit board 200. For instance, in some implementations, the width of the slot antenna 400 can range from about 0.5 millimeters to about 10 millimeters.

The slot antenna 400 can be operable at a plurality of different frequency bands. For instance, the slot antenna 400 can be operable at one or more Global Navigation Satellite System (GNSS) frequency bands. In some implementations, the one or more GNSS frequency bands can include one or more GPS frequency bands. The one or more GPS frequency bands can include at least one of a first GPS frequency band ranging from about 1164 Megahertz (MHz) to about 1189 MHz, a second GPS frequency band ranging from about 1563 MHz to about 1587 MHz, and a third GPS frequency band ranging from about 1215 MHz to about 1240 MHz. Furthermore, in addition to the one or more GPS frequency bands, the slot antenna 400 can be configured to radiate at one or more frequency bands associated with cellular communications (e.g., 4G, 5G) or wireless local area communications. It should be understood however that the slot antenna 400 can be operable at frequency bands associated with any suitable communication standard.

In some implementations, the slot antenna 400 can include a first grounding contact 402 and a second grounding contact 404. The first grounding contact 402 can be coupled between the conductive housing 110 and a first location on the perimeter (e.g., ground keep-out region 206) of the printed circuit board 200. Conversely, the second grounding contact 404 can be coupled between the conductive housing 110 and a second location on the perimeter (e.g., ground keep-out region 206) of the printed circuit board 200. In some implementations, the first location and the second location can correspond to opposing sides of the printed circuit board 200. It should be understood however that the first grounding contact 402 and the second grounding contact 404 can be coupled to the perimeter of the printed circuit board 200 at any suitable location to adjust the length of the slot antenna 400. For instance, the first grounding contact 402 and the second grounding contact 404 can be positioned closer to one another to shorten the slot antenna 400. Alternatively, the first grounding contact 402 and the second grounding contact 404 can be positioned to lengthen the slot antenna 400.

In some implementations, the wearable computing device 100 can include a shielding can 210. As shown, the shielding can 210 can be positioned over a portion of the printed circuit board 200. For instance, the shielding can 210 can be positioned over the portion of the printed circuit board 200 that includes one or more electrical circuits disposed on the printed circuit board 200. In this manner, the shielding can 210 can electrically shield the one or more electrical circuits from electromagnetic interference (EMI).

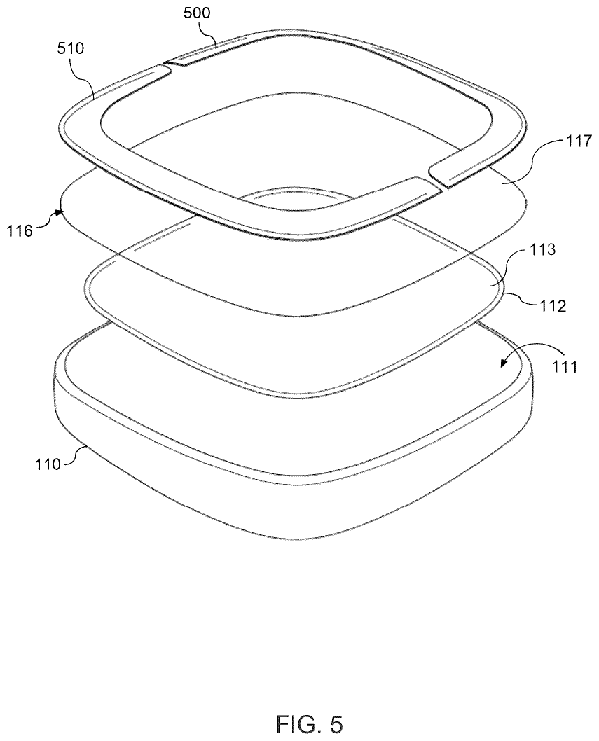

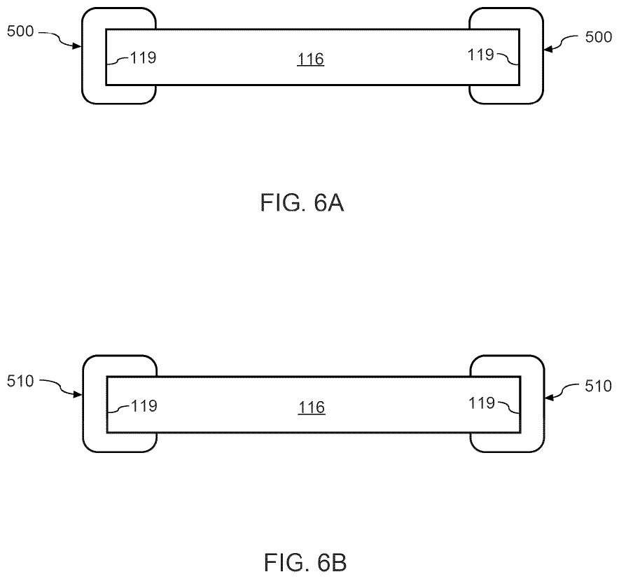

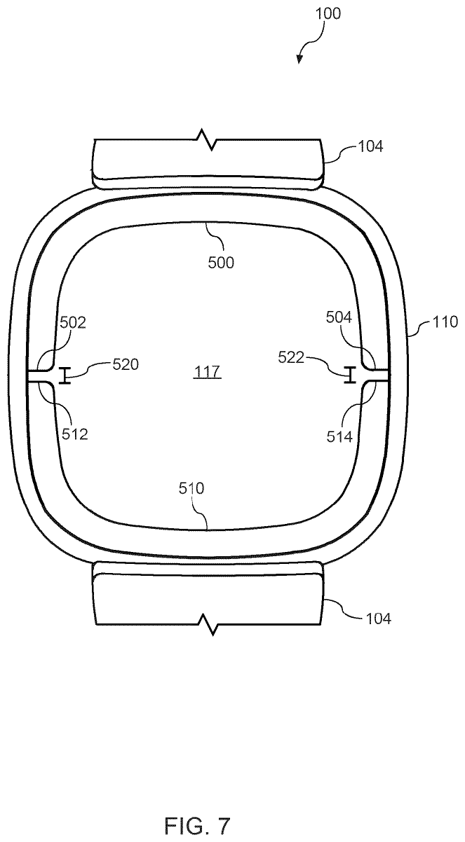

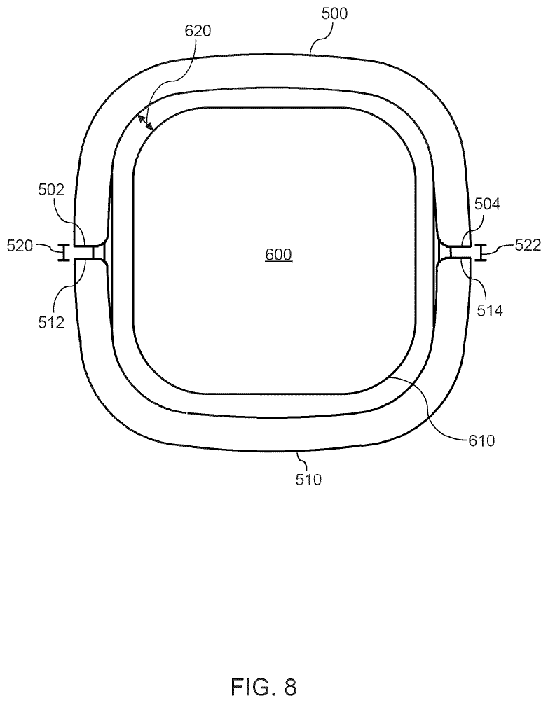

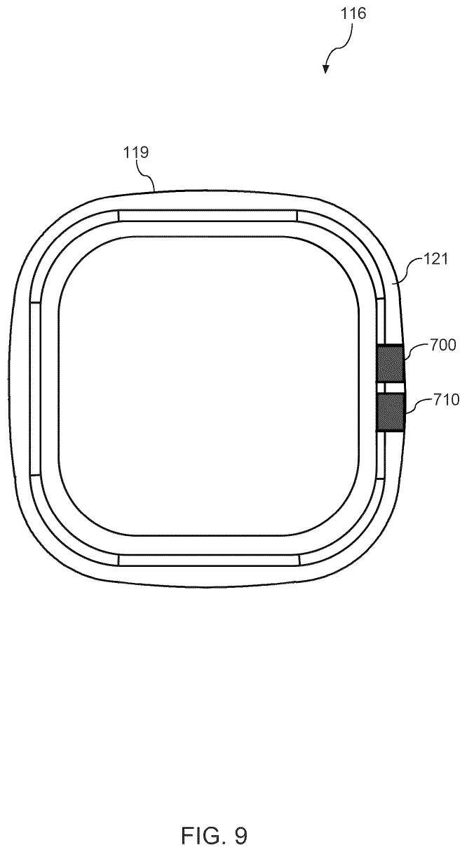

Referring now to FIGS. 5, 6A, 6B, and 7 through 9, the one or more biometric sensor electrodes 118 (FIG. 1) of the wearable computing device 100 can include a first biometric sensor electrode 500 and a second biometric sensor electrode 510. In alternative implementations, the wearable computing device 100 can include more than two biometric sensor electrodes.

As shown, the first biometric sensor electrode 500 and the second biometric sensor electrode 510 can be positioned on the top surface 117 of the cover 116. For instance, the first biometric sensor electrode 500 and the second biometric sensor electrode 510 can each be partially positioned on a portion of the top surface 117 of the cover 116. More particularly, the portion of the top surface 117 can include an outermost portion (e.g., periphery) of the top surface 117. In this manner, the first biometric sensor electrode 500 and the second biometric sensor electrode 510 can eliminate the need for having a separate element, such as a bezel, to frame the display screen 112.

The first biometric sensor electrode 500 and the second biometric sensor electrode 510 can cover at least a portion (e.g., a dead band) of an area of the display screen 112 that surrounds an active display area 600 of the display screen 112. More particularly, the area that is at least partially covered by the first biometric sensor electrode 500 and the second biometric sensor electrode 510 can extend from an edge 610 of the active display area 600 to the periphery 119 of the cover 116.

In some implementations, a gap 620 can be defined between the edge 610 of the active display area 600 and the biometric sensor electrodes (e.g., first biometric sensor electrode 500 and second biometric sensor electrode 510). For instance, in some implementations, the width of the gap 620 can range from about 0.5 millimeters to about 3 millimeters. It should be appreciated that the slot antenna 400 (FIG. 4) can radiate through the gap 620. As will be discussed below in more detail, a width of the gap 620 can vary depending on a sheet resistance of the biometric sensor electrodes (e.g., first biometric sensor electrode 500 and second biometric sensor electrode 510).

Furthermore, since the first biometric sensor electrode 500 and the second, biometric sensor electrode 510 are positioned on the top surface 117 of the cover 116, the first biometric sensor electrode 500 and the second biometric sensor electrode 510 can allow for on-demand measurement of biometrics (e.g. electrocardiogram, electrodermal activity, etc.) of the user wearing the wearable computing device 100. For instance, in some implementations, the user can contact (e.g., touch) the first biometric sensor electrode 500 to obtain an on-demand electrocardiogram reading. Alternatively, or additionally, the user can contact (e.g. touch) both the first biometric sensor electrode 500 and the second biometric sensor electrode 510 to obtain an on-demand electrodermal activity reading.

In some implementations, the first biometric sensor electrode 500 and the second biometric sensor electrode 510 can be spaced apart from one another on the top surface 117 of the cover 116. For instance, a first gap 520 can be defined between a first end 502 of the first biometric sensor electrode 500 and a first end 512 of the second biometric sensor electrode 510. Additionally, a second gap 522 can be defined between a second end 504 of the first biometric sensor electrode 500 and a second end 514 of the second biometric sensor electrode 510. In some implementations, a width of the first gap 520 and a width of the second gap 522 can be the same. For instance, in some implementations, the width of the first gap 520 and the width of the second gap 522 can range from about 0.5 millimeters to about 2 millimeters. In alternative implementations, the width of the first gap 520 can be different (e.g., narrower, wider) than the width of the second gap 522.

The first biometric sensor electrode 500 and the second biometric sensor electrode 510 can each at least partially wrap around the periphery 119 of the cover 116 to contact (e.g., touch) one or more electrical contacts on a bottom surface 121 of the cover 116. For instance, in some implementations, the bottom surface 121 of the cover 116 can include a first electrical contact 700 and a second electrical contact 710. In such implementations, the first biometric sensor electrode 500 can wrap around a first portion of the periphery 119 of the cover 116 to contact the first electrical contact 700 on the bottom surface 121 of the cover 116. Furthermore, the second biometric sensor electrode 510 can wrap around a second portion of the periphery 119 of the cover 116 to contact the second electrical contact 710 on the bottom surface 121 of the cover 116.

It should be understood that the second portion of the periphery 119 of the cover 116 is different than the first portion of the periphery 119 of the cover 116. For instance, in some implementations, the first portion of the periphery 119 can correspond to an upper half of the cover 116. Furthermore, in such implementations, the second portion of the periphery 119 can correspond to a lower half of the cover 116. It should also be understood that, in some implementations, the bottom surface 121 of the cover 116 can include more electrical contacts. For instance, in some implementations, the bottom surface 121 of the cover 116 can include multiple electrical contacts to which the first biometric sensor electrode 500 can be connected. Alternatively, or additionally, the bottom surface 121 of the cover 116 can include multiple electrical contacts to which the second biometric sensor electrode 510 can be connected.

In some implementations, the top surface 117 of the cover 116 and the bottom surface 121 of the cover 116 can each be a substantially flat surface. Alternatively, or additionally, the periphery 119 of the cover 116 can be curved. It should be understood, however, that the cover 116 can have any suitable shape. It should also be understood that, in some implementations, the first biometric sensor electrode 500 and the second biometric sensor electrode 510 can be electrically coupled to the printed circuit board 200 via a flexible printed circuit. For instance, in some implementations, the flexible printed circuit can be coupled between the printed circuit board 200 and the one or more electrical contact (e.g., first electrical contact 700, second electrical contact 710) on the bottom surface 121 of the cover 116. In this manner, signals associated with biometric measurements can be communicated to one or more circuits on the printed circuit board 200 via the flexible printed circuit.

In some implementations, at least a portion of at least one of the first biometric sensor electrode 500 or the second biometric sensor electrode 510 can include a physical vapor deposition (PVD) defined coating. For instance, in some implementations, a first PVD defined coating can cover at least a portion of the first biometric sensor electrode 500 that is positioned on the top surface 117 of the cover 116. Alternatively, or additionally, the first PVD defined coating can cover at least a portion of the first biometric sensor electrode 500 that is positioned on the bottom surface 121 of the cover 116. It should be understood that the first PVD defined coating can cover any portion of the first biometric sensor electrode 500. For instance, in some implementations, the first PVD defined coating can completely cover the first biometric sensor electrode 500.

In some implementations, a second PVD defined coating can cover at least a portion of the second biometric sensor electrode 510 that is positioned on the top surface 117 of the cover 116. Alternatively, or additionally, the second PVD defined coating can cover at least a portion of the second biometric sensor electrode 510 that is positioned on the bottom surface 121 of the cover 116. It should be understood that the second PVD defined coating can cover any portion of the second biometric sensor electrode 510. For instance, in some implementations, the first PVD defined coating can completely cover the second biometric sensor electrode 510.

It should be understood that placement of the first biometric sensor electrode 500 and the second biometric sensor electrode 510 on the cover 116 can degrade performance of the slot antenna 400 (FIG. 4). For instance, the first biometric sensor electrode 500 and the second biometric sensor electrode 510 can interfere with or degrade a radiation pattern of the slot antenna 400. As will be discussed below, the first biometric sensor electrode 500 and the second biometric sensor electrode 510 can be configured so as to reduce or eliminate a degradation in performance of the slot antenna 400 due to placement of the first biometric sensor electrode 500 and the second biometric sensor electrode 510 on the cover 116.

In some implementations, the first biometric sensor electrode 500 and the second biometric sensor electrode 510 can each have a first sheet resistance value such that the at least one of the first biometric sensor electrode 500 and the second biometric sensor electrode 510 are each radio frequency transparent for a range of frequencies at which the slot antenna. 400 (FIG. 4) is operable. For instance, in some implementations, the first sheet resistance value can include any sheet resistance value that is greater than about 2000 ohms per square for the range of frequencies (e.g., about 1 Gigahertz to about 3 Gigahertz) at which the slot antenna 400 (FIG. 4) is operable. In this manner, degradation in the performance of the slot antenna 400 over the range of frequencies due to placement of first biometric sensor electrode 500 and the second biometric sensor electrode 510 on the cover 116 can be reduced or eliminated, because the first biometric sensor electrode 500 and the second biometric sensor electrode 510 are each radio frequency transparent for the range of frequencies at which the slot antenna 400 is operable.

In some implementations, the first biometric sensor electrode 500 and the second biometric sensor electrode 510 can each have a second sheet resistance value that is less than the first sheet resistance value so that the first biometric sensor electrode 500 and the second biometric sensor electrode 510 can support radio frequency currents for loading the slot antenna 400 (FIG. 4) over the range of frequencies at which the slot antenna 400 is operable. More particularly, the first biometric sensor electrode 500 and the second biometric sensor electrode 510 can support radio frequency currents for loading the slot antenna 400 without causing losses associated with the radiation of the slot antenna 400. For instance, in some implementations, the second sheet resistance value can be less than about 200 ohms per square for the range of frequencies at which the slot antenna. 400 is operable. In this manner, degradation in the performance of the slot antenna over the range of frequencies due to placement of the one or more biometric sensor electrodes on the cover can be reduced or eliminated, because the first biometric sensor electrode 500 and the second biometric sensor electrode 510 each support radio frequency currents for loading the slot antenna 400 over the range of frequencies at which the slot antenna 400 is operable. In some implementations, the second sheet resistance value can be less than 100 ohms per square, such as from about 20 ohms per square to about 40 ohms per square.

In implementations in which the first biometric sensor electrode 500 and the second biometric sensor electrode 510 each have the second sheet resistance value to support radio frequency currents for loading the slot antenna 400, the width of the gap 620 is greater than the width of the gap 620 in implementations in which the first biometric sensor electrode 500 and the second biometric sensor electrode 510 each have the first resistance value such that the first biometric sensor electrode 500 and the second biometric sensor electrode 510 are at least partially radio frequency transparent. For instance, in some implementations the width of the gap 620 must be greater than 1 millimeter in order to reduce or eliminate the likelihood of the first biometric sensor electrode 500 and the second biometric sensor electrode 510 interfering with the radiation pattern associated with the slot antenna 400 (FIG. 4).

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A wearable computing device comprising:
a conductive housing;
a printed circuit board at least partially disposed within the conductive housing;
a slot antenna defined by a gap between the conductive housing and the printed circuit board;
a display screen electrically coupled to the printed circuit board;
a cover positioned on the display screen, the cover comprising a top surface and a bottom surface; and
a biometric sensor electrode positioned partially on the top surface of the cover, the biometric sensor electrode at least partially wrapping around a periphery of the cover.

2. The wearable computing device of claim 1, wherein the biometric sensor electrode has a sheet resistance such that the biometric sensor electrode is at least partially radio frequency transparent at a frequency at which the slot antenna is operable.

3. The wearable computing device of claim 2, wherein the sheet resistance is about 3000 ohms per square for the frequency at which the slot antenna is operable.

4. The wearable computing device of claim 1, wherein the biometric sensor electrode has a sheet resistance such that the biometric sensor electrode supports radio frequency currents for loading the slot antenna at a frequency at which the slot antenna is operable.

5. The wearable computing device of claim 4, wherein the sheet resistance is less than about 200 ohms per square for the frequency at which the slot antenna is operable.

6. The wearable computing device of claim 1, wherein a gap is defined between the biometric sensor electrode and an active display area of the display screen.

7. The wearable computing device of claim 6, wherein a width of the gap ranges from about 0.5 millimeters to about 3 millimeters.

8. The wearable computing device of claim 2, wherein the frequency ranges from 0.6 Gigahertz to 10 Gigahertz.

9. The wearable computing device of claim 1, wherein the cover comprises an optically transparent material.

10. The wearable computing device of claim 9, wherein the optically transparent material comprises a glass material.

11. The wearable computing device of claim 1, further comprising:
an electrical contact disposed on the bottom surface of the cover, the biometric sensor electrode contacting the electrical contact.

12. The wearable computing device of claim 1, further comprising:
a physical vapor deposition (PVD) defined coating covering at least a portion of the biometric sensor electrode.

13. The wearable computing device of claim 12, wherein the portion of the biometric sensor electrode comprises at least one of a first portion of the biometric sensor electrode or a second portion of the biometric sensor electrode, the first portion positioned on the top surface of the cover, the second portion positioned on the bottom surface of the cover.

14. The wearable computing device of claim 1, wherein:
the top surface and the bottom surface each comprise a flat surface; and
the periphery of the cover comprise a curved surface.

15. A wearable computing device comprising:
a conductive housing;
a printed circuit board at least partially disposed within the conductive housing;
a slot antenna defined by a gap between the conductive housing and the printed circuit board;
a display screen electrically coupled to the printed circuit board;
a cover positioned on the display screen, the cover comprising a top surface and a bottom surface; and
a first biometric sensor electrode and a second biometric sensor electrode, the first biometric sensor electrode and the second biometric sensor electrode each positioned partially on the top surface of the cover such that the first biometric sensor electrode and the second biometric sensor electrode are spaced apart from one another, the first biometric sensor electrode wrapping around a first portion of a periphery of the cover, the second biometric sensor electrode wrapping around a second portion of the periphery of the cover,
wherein the first biometric sensor electrode has a sheet resistance such that the first biometric sensor electrode is at least partially radio frequency transparent at a frequency at which the slot antenna is operable.

16. The wearable computing device of claim 15, wherein the sheet resistance is greater than 2000 ohms per square at the frequency at which the slot antenna is operable.

17. The wearable computing device of claim 15, wherein the frequency ranges from 0.6 Gigahertz to 10 Gigahertz.

18. A wearable computing device comprising:
a conductive housing;
a printed circuit board at least partially disposed within the conductive housing;
a slot antenna defined by a gap between the conductive housing and the printed circuit board;
a display screen electrically coupled to the printed circuit board;
a cover positioned on the display screen, the cover comprising a top surface and a bottom surface; and
a first biometric sensor electrode and a second biometric sensor electrode, the first biometric sensor electrode and the second biometric sensor electrode positioned partially on the top surface of the cover such that the first biometric sensor electrode and the second biometric sensor electrode are spaced apart from one another, the first biometric sensor electrode wrapping around a first portion of a periphery of the cover, the second biometric sensor electrode wrapping around a second portion of the periphery of the cover,
wherein the first biometric sensor electrode has a sheet resistance such that the first biometric sensor electrode supports radio frequency currents for loading the slot antenna at a frequency at which the slot antenna is operable.

19. The wearable computing device of claim 18, wherein a width of a gap defined between the first biometric sensor electrode and an edge of an active display area of the display screen ranges from 0.5 millimeters to about 3 millimeters.

20. The wearable computing device of claim 18, wherein the sheet resistance is less than 200 ohms per square at the frequency at which the slot antenna is operable.
